(19) United States
(12) Patent Application Publication
(10) Pub. No.: US 2025/0333376 A1
(43) Pub. Date: Oct. 30, 2025

(54) CRYSTAL FORMS OF BMX AND PREPARATION THEREOF

(71) Applicant: Novelwise Pharmaceutical Corporation, Taipei City (TW)

(72) Inventors: Zhong-Yang HUANG, Taipei City (TW); Chia-Chung HOU, Taipei City (TW)

(73) Assignee: Novelwise Pharmaceutical Corporation, Taipei City (TW)

(21) Appl. No.: 19/187,167

(22) Filed: Apr. 23, 2025

Related U.S. Application Data
(60) Provisional application No. 63/638,164, filed on Apr. 24, 2024.

(51) Int. Cl. C07C 259/06

(52) U.S. Cl. CPC C07C 259/06; C07B 2200/13

(57) ABSTRACT

The present invention relates to some crystal forms for a cinnamic compound, BMX, which is an inhibitor of histone deacetylase (HDAC), useful as an agent for the prevention or treatment of diseases associated with HDAC, including for treating tumor or cell proliferative diseases, diabetes mellitus, or neurodegenerative diseases such as Alzheimer's disease, Huntington's disease, Spinocerebellar Ataxias (SCA) and human spinal muscular atrophy (SMA). Also provided are a method for preparing the crystal forms and pharmaceutical compositions comprising the crystal forms.

### CROSS REFERENCE

This Non-provisional application claims the priority under 35 U.S.C. § 119(e) on U.S. Patent Provisional Application No. 63/638,164 filed on Apr. 24, 2024, the entire contents of which are hereby incorporated by reference.

### FIELD OF THE INVENTION

The present invention pertains to crystal forms of a compound BMX (C₂₃H₂₇NO₅), and the preparation method thereof.

### BACKGROUND OF THE INVENTION

Compound BMX (C₂₃H₂₇NO₅) is a cinnamic compound which is an inhibitor of histone deacetylase (HDAC), and useful as an agent for the prevention or treatment of diseases associated with HDAC. The Compound BMX was designed and synthesized by Naturewise Biotech and Medicals Corporation (Taipei, Taiwan). The chemical structure of Compound BMX is shown below (also see FIG. 1), as described in U.S. Pat. No. 7,994,357B2:

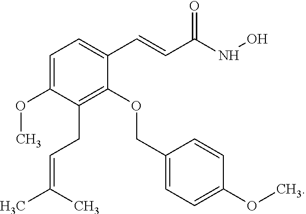 [SMILES: COc1ccc(COc2c(/C=C/C(=O)NO)ccc(OC)c2CC=C(C)C)cc1]

As shown in U.S. Pat. No. 7,994,357B2, Compound BMX is used as an agent for enhancing the neurite outgrowth, in particular, an agent for treating tumor or cell proliferative diseases, diabetes mellitus, or neurodegenerative diseases such as Alzheimer's disease, Huntington's disease, Spinocerebellar Ataxias (SCA) and human spinal muscular atrophy (SMA). Although the preparation method of Compound BMX was disclosed in U.S. Pat. No. 7,994,357 B2, it was not indicated whether the product is a crystalline substance, nor did it indicate whether there was polymorphism for Compound BMX.

Crystal form is one of the key factors that affect the quality, therapeutic efficacy and formulation processing performance of the drug substance. Polymorphism refers to the phenomenon that one compound may form two or more molecular spatial arrangements by controlling the conditions, and is a key factor to affect the quality of a drug product. Different crystal structures of the same compound, despite having identical chemical composition, result in differences in their external morphology, physicochemical properties and biological activities. Therefore, different crystal forms of a drug substance tend to be different in solubility, storage stability, hygroscopicity, density and bioavailability, and directly affect the quality of its pharmaceutical formulation and the absorption in the human body. Hence, it is of great significance to investigate the polymorphism of a drug substance and the preparation methods for different crystal forms.

Until now, it is desirable to develop new crystal forms of Compound BMX.

### SUMMARY OF THE INVENTION

Accordingly, the present invention provides novel crystal forms of Compound BMX, wherein Compound BMX stands for a compound with the chemical structure displayed below (see FIG. 1):

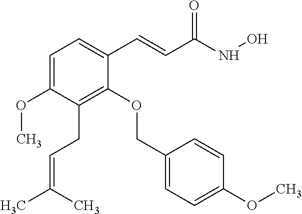 [SMILES: COc1ccc(COc2c(/C=C/C(=O)NO)ccc(OC)c2CC=C(C)C)cc1]

In one aspect, the present invention provides some crystal forms of Compound BMX, including the crystal forms A, B, C, D, E, F and G.

In one embodiment of the present invention, Crystal Form A of the Compound BMX is characterized in an X-ray powder diffraction pattern (XRPD) having peaks 2θ of about 4.58°, 7.58°, 9.14°, 11.30°, 12.41°, 13.71°, 15.05°, 15.41°, 16.27°, 16.97°, 18.44°, 19.16°, 19.51°, 19.87°, 20.49°, 22.71°, 22.92°, 23.33°, 23.86°, 24.92°, 25.55°, 26.36°, 27.58°, 28.00°, 28.48°, 28.77°, 29.38°, and 30.32°, and an infrared spectrum having characteristic absorption peaks at about 3250, 2909, 2837, 1643, 1590, 1515, 1484, 1462, 1250, 1173, 1088, 991, 823, 806, 796, 516 and 481 cm⁻¹. The Crystal Form A of Compound BMX is also characterized by a Differential Scanning Calorimetry (DSC) pattern having a sharp single melt at T peak=137.8° C. and a recrystallization event at T peak=155.1° C.

In one example of the invention, the Crystal Form A of Compound BMX is prepared by the method of the steps of
dissolving Compound BMX in ethyl acetate;
slowly adding n-hexane with stirring and cooling it to a room temperature to obtain a solution;
cooling the solution to 0˜5° C. for accelerating precipitation to obtain the Crystal Form A.

In another embodiment of the present invention, Crystal Form B of Compound BMX is characterized in an X-ray powder diffraction (XRPD) pattern having peaks at 2θ of about 7.19°, 8.86°, 9.36°, 11.40°, 12.17°, 13.32°, 14.08°, 14.88°, 15.43°, 16.30°, 16.72°, 17.83°, 18.91°, 19.81°, 23.13°, 23.75°, 27.01°, 28.13°, 30.10°, and 31.42°. The Crystal Form B is also characterized in a Differential Scanning Calorimetry (DSC) pattern having a sharp single melt at T peak=132° C. and a recrystallization event at T peak=89° C.

In one example of the present invention, Crystal Form B is prepared by a method of the steps of:
dissolving Compound BMX in dichloromethane and/or in methanol to obtain a solution;
fast evaporating the solution obtained in the above step to obtain an amorphous material of Compound BMX;
redissolving the amorphous material of Compound BMX in MeOH and water to obtain the Crystal Form B.

In one embodiment of the present invention provides, Crystal Form C of Compound BMX is characterized in an X-ray powder diffraction (XRPD) pattern having peaks at 2θ of about 14.08°, 16.17°, 16.30°, 16.60°, 18.08°, 18.43°, 18.71°, 19.55°, 21.13°, 23.38°, 24.35°, 24.64°, 26.13°, 27.65°, and 32.33°.

In one example of the present invention, Crystal Form C is prepared by a method of the steps of:
dissolving the Crystal Form A of Compound BMX in DMSO and EtOH slurry at 50° C. to obtain the Crystal Form C.

In one more embodiment of the present invention, Crystal Form D of Compound BMX is characterized in an X-ray powder diffraction (XRPD) pattern having peaks at 2θ of about 4.77°, 18.65°, 19.10°, 19.32°, 19.55°, 20.17°, 20.18°, 20.36°, 20.84°, 21.92°, 22.54°, 22.98°, 23.16°, 24.02°, 24.25°, 24.38°, 24.67°, 25.23°, 25.89°, 26.86°, 33.64°. Crystal Form D is also characterized a Differential Scanning Calorimetry (DSC) pattern with melt at T peak=133.0° C.

In one example of the present invention, Crystal Form D is prepared by a method of the steps of:
dissolving an amorphous material of Compound BMX in 2-MeTHF/toluene to obtain a slurry;
shaking the slurry at room temperature to obtain Crystal Form D.

In one yet embodiment of the present invention, Crystal Form E of Compound BMX is characterized in an X-ray powder diffraction (XRPD) pattern having peaks at 2θ of about 4.61°, 7.60°, 9.13°, 12.40°, 15.42°, 16.27°, 16.56°, 16.95°, 19.85°, 21.49°, 21.84°, 22.48°, 23.19°, 23.85°, 24.56°, 25.49°, 25.99°, 28.42°, and 34.26°. Crystal Form E is also characterized a Differential Scanning Calorimetry (DSC) pattern with melt at T peak=127.1° C.

In one example of the present invention, Crystal Form E is prepared by a method of the steps of:
dissolving an amorphous material of Compound BMX in DMSO and ethanol to obtain a slurry mixture;
shaking the slurry mixture at 50° C. to obtain the Crystal Form E.

In one further yet embodiment of the present invention, Crystal Form F of Compound BMX is characterized in an X-ray powder diffraction (XRPD) pattern having peaks at 2θ of about 7.32°, 9.37°, 11.47°, 12.23°, 13.41°, 14.07°, 14.93°, 15.54°, 16.38°, 16.74°, 17.07°, 17.86°, 18.18°, 18.90°, 19.90°, 21.87°, 22.61°, 23.21°, 23.72°, 27.18°, and 28.16°. Crystal Form F is also characterized a Differential Scanning Calorimetry (DSC) pattern with melt at T peak=133.5° C. and 139.2° C.

In one example of the present invention, Crystal Form F is prepared by a method comprising the steps of:
dissolving an amorphous powder of compound BMX in 1,3-Dioxolane and keeping it at 50° C. to obtain a slurry;
cooling the slurry to room temperature and adding water as an anti-solvent to obtain the Crystal Form F.

In one further embodiment of the present invention, Crystal Form G of Compound BMX is characterized in an X-ray powder diffraction (XRPD) pattern having peaks at 2θ of about 8.92°, 9.27°, 11.94°, 13.58°, 13.78°, 14.94°, 15.92°, 17.68°, 19.21°, 20.19°, 21.65°, 22.99°, 27.75°, and 32.55°. Crystal Form G is also characterized a Differential Scanning Calorimetry (DSC) pattern with melt at T peak=132.6° C.

In one example of the present invention, Crystal Form G is prepared by a method of the steps of:
dissolving an amorphous material of Compound BMX in methyl isobutyl ketone at 50° C. to obtain a slurry;
cooling the slurry to room temperature and adding heptane as an anti-solvent to obtain the Crystal Form G.

The present invention also provides a pharmaceutical composition comprising any one of the crystal forms of Compound BMX according to the invention and a pharmaceutically acceptable carrier.

According to the invention, the pharmaceutical composition may be formulated with a pharmaceutically acceptable carrier by standard, conventional or commonly used methods. The term “pharmaceutically acceptable carrier” as used herein refers to an excipient, extender, disintegrant, binder, lubricant, colorant, diluent, wetting agent, surfactant, dispersant, buffer agent, preservative, solubilizer, antiseptic, flavoring agent, soothing agent, and stabilizer. One example of the pharmaceutical acceptable carrier is a buffer solution or a saline.

It is to be understood that both the foregoing general description and the following detailed description are exemplary and explanatory only and are not restrictive of the invention.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, they are shown in the drawings embodiments which are presently preferred.

In the drawings:

FIG. 1 shows the chemical structure of Compound BMX.

FIGS. 2A-2G show the XRPD patterns of various crystal forms of Compound BMX; wherein FIG. 2A shows the XRPD pattern of Crystal Form A, FIG. 2B shows the XRPD pattern of Crystal Form B, FIG. 2C shows the XRPD pattern of Crystal Form C, FIG. 2D shows the XRPD pattern of Crystal Form D, FIG. 2E shows the XRPD pattern of Crystal Form E, FIG. 2F shows the XRPD pattern of Crystal Form F, and FIG. 2G shows the XRPD pattern of Crystal Form G.

FIG. 3 shows the DSC thermogram of the Crystal Form A of Compound BMX.

FIG. 4 shows the TGA/DSC thermogram of the Crystal Form A of Compound BMX.

FIG. 5 shows the microscopy image of the Crystal Form A of Compound BMX.

FIG. 6 shows DVS sorption and de-sorption plot of the Crystal Form A of Compound BMX.

FIG. 7 shows the DVS isotherm plot of the Crystal Form A of Compound BMX.

FIG. 8 shows the XRPD overlay of the Crystal Form A of Compound BMX (lower), evaporation experiment in methanol (middle) and evaporation experiment in dichloromethane (upper).

FIG. 9 shows the TGA/DSC thermogram of the amorphous material of Compound BMX after evaporation from dichloromethane.

FIG. 10 shows the TGA/DSC thermogram of the amorphous material of Compound BMX after evaporation from methanol.

FIG. 11 shows the XRPD overlay of the Crystal Forms A, B, C and D of Compound BMX.

FIGS. 12A-12B show the XRPD overlay of the crystal forms of Compound BMX. FIG. 12A shows the XRPD overlay of Crystal Forms A, B, C, D and E of Compound BMX; FIG. 12B shows the XRPD overlay of Crystal Forms A, F, and G of Compound BMX.

FIG. 13 shows the XRPD overlay of the Crystal Form A (upper) and competitive slurry experiment in 2-propanol (lower) of Compound BMX.

FIG. 14 shows the XRPD overlay of Crystal Form B of Compound BMX, which was tested by stability experiments in water.

FIG. 15 shows the XRPD overlay of the Crystal Form B (lower) in stability experiments with seedings of Form A (upper and middle).

FIG. 16 provides an image showing the stress stability results of the Crystal Form A and the amorphous material of Compound BMX.

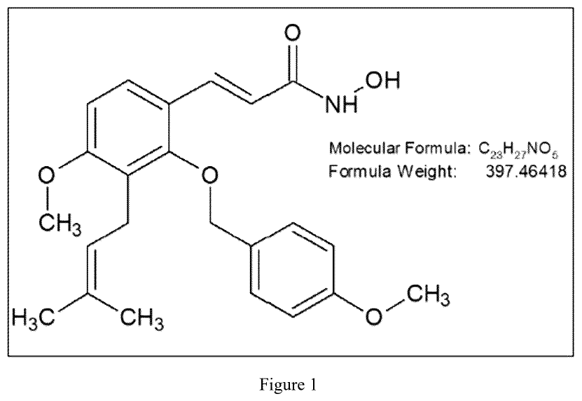

### DETAILED DESCRIPTION OF THE INVENTION

Unless defined otherwise, all technical and scientific terms used herein have the same meaning as commonly understood by a person skilled in the art to which this invention belongs.

The present invention provides seven crystal forms of Compound BMX, including Crystal Forms A, B, C, D, E, F and G.

The Crystal Form A of Compound BMX is present as white, non-hygroscopic powder with a single sharp melt at T=137.8° C. and needles of about 20 μm in its microscopy study, which shows no mass loss before and during TGA test. The Crystal Form A is characterized in X-ray powder diffraction (XRPD) pattern having characteristic peaks at 2θ of about 4.58°, 7.58°, 9.14°, 11.30°, 12.41°, 13.71°, 15.05°, 15.41°, 16.27°, 16.97°, 18.44°, 19.16°, 19.51°, 19.87°, 20.49°, 22.71°, 22.92°, 23.33°, 23.86°, 24.92°, 25.55°, 26.36°, 27.58°, 28.00°, 28.48°, 28.77°, 29.38°, and 30.32°; and the infrared spectrum having characteristic absorption peaks at about 3250, 2909, 2837, 1643, 1590, 1515, 1484, 1462, 1250, 1173, 1088, 991, 823, 806, 796, 516 and 481 cm⁻¹. The form is also characterized in the differential scanning calorimetry (DSC) pattern having a sharp single melt at 137.8° C. and a recrystallization event at 155.1° C.

In one example of the present invention, the Crystal Form A is prepared by the method of the steps of
dissolving Compound BMX in ethyl acetate to obtain a solution;
slowly adding in n-hexane with stirring and decreasing it to a room temperature;
reducing the temperature of the solution to 0˜5° C. for accelerating precipitation to obtain the Crystal Form A.

The Crystal Form B of Compound BMX is present as white powder with a recrystallization event at 89° C. followed by the original melt at 132° C., which is very stable in water once it is formed. The crystal Form B is characterized in X-ray powder diffraction (XRPD) pattern having peaks at 2θ of about 7.19°, 8.86, 9.36°, 11.40°, 12.17°, 13.32°, 14.08°, 14.88°, 15.43°, 16.30°, 16.72°, 17.83, 18.91°, 19.81°, 23.13°, 23.75°, 27.01°, 28.13°, 30.10°, and 31.42°. The form is also characterized in a differential scanning calorimetry (DSC) pattern having a sharp single melt at 132° C. and a recrystallization event at 89° C.

In one example of the present invention, the Crystal Form B is prepared by a method of the steps of:
dissolving Compound BMX in dichloromethane and/or in methanol to obtain a solution;
fast evaporating the solution as obtained in the above step to obtain an amorphous material of Compound BMX; and
redissolving the amorphous in MeOH, water to obtain the Crystal Form B.

In the invention, the Crystal Form B of Compound BMX with a purity >99% was obtained, which remained stable in water.

The present invention also provides Crystal Form C of Compound BMX, which is characterized in its X-ray powder diffraction method (XRPD) pattern having peaks at 2θ of about 14.08°, 16.17°, 16.30°, 16.60°, 18.08°, 18.43°, 18.71°, 19.55°, 21.13°, 23.38°, 24.35°, 24.64°, 26.13°, 27.65°, and 32.33°.

In one example of the present invention, Crystal Form C is prepared by a method of the steps of:
dissolving Compound BMX Form A in DMSO and EtOH slurry at 50° C. to obtain the Crystal Form C.

Further, the present invention provides Crystal Form D of Compound BMX, which is characterized in X-ray powder diffraction method (XRPD) pattern having peaks at 2θ of about 4.77°, 18.65°, 19.10°, 19.32°, 19.55°, 20.17°, 20.18°, 20.36°, 20.84°, 21.92°, 22.54°, 22.98°, 23.16°, 24.02°, 24.25°, 24.38°, 24.67°, 25.23°, 25.89°, 26.86°, 33.64°. The Crystal Form D is also characterized in the Differential Scanning Calorimetry (DSC) pattern with melt at T peak=133.0° C.

In one example of the present invention, the Crystal Form D is prepared by a method of the steps of:
dissolving amorphous material of Compound BMX in 2-MeTHF/toluene;
shaking slurry at room temperature to obtain the Crystal Form D.

In addition, the present invention provides Crystal Form E of Compound BMX, which is characterized in X-ray powder diffraction method (XRPD) pattern having peaks at 2θ of about 4.61°, 7.60°, 9.13°, 12.40°, 15.42°, 16.27°, 16.56°, 16.95°, 19.85°, 21.49°, 21.84°, 22.48°, 23.19°, 23.85°, 24.56°, 25.49°, 25.99°, 28.42°, and 34.26°. The Crystal Form E is also characterized in the Differential Scanning Calorimetry (DSC) pattern with melt at T peak=127.1° C.

In one example of the present invention, the crystal Form E is prepared by a method of the steps of:
dissolving amorphous material of Compound BMX in DMSO and ethanol;
then slurry the mixture at 50° C. to obtain the crystal Form E.

The present invention also provides Crystal Form F of Compound BMX, which is characterized in X-ray powder diffraction method (XRPD) pattern having peaks at 2θ of about 7.32°, 9.37°, 11.47°, 12.23°, 13.41°, 14.07°, 14.93°, 15.54°, 16.38°, 16.74°, 17.07°, 17.86°, 18.18°, 18.90°, 19.90°, 21.87°, 22.61°, 23.21°, 23.72°, 27.18°, and 28.16°. The crystal Form F is also characterized in the Differential Scanning Calorimetry (DSC) pattern with melt at T peak=133.5° C. and 139.2° C.

In one example of the present invention, the Crystal Form F is prepared by a method of the steps of:
Compound BMX amorphous powder was dissolved in 1,3-Dioxolane at kept at 50° C.;
cooling to room temperature and added water as anti-solvent to obtain the crystal Form F.

In addition, the present invention provides Crystal Form G of Compound BMX, which is characterized in X-ray powder diffraction method (XRPD) pattern having peaks at 2θ of about 8.92°, 9.27°, 11.94°, 13.58°, 13.78°, 14.94°, 15.92°, 17.68°, 19.21°, 20.19°, 21.65°, 22.99°, 27.75°, and 32.55°. The Crystal Form G is also characterized in the Differential Scanning Calorimetry (DSC) pattern with melt at T peak=132.6° C.

In one embodiment of the present invention, the Crystal Form G is prepared by a method of the steps of:
dissolving amorphous Compound BMX in methyl isobutyl ketone at 50° C.;
cooling to room temperature and added heptane as anti-solvent to obtain the Crystal Form G.

As found in the present invention, the Crystal Form A was the most stable form, and the Crystal Form B was stable. The crystal Forms C-E appeared in the screening step only, and during reproduction, the Crystal Form D was converted into the Crystal Forms F and G, and Crystal Form E into Crystal Form B, Crystal Form C was unstable and converted into Crystal Form D soon.

The present invention is further illustrated by the following examples, which are provided for the purpose of demonstration rather than limitation.

### EXAMPLES

### Example 1. XRPD Pattern Examination of Crystal Form A of Compound BMX

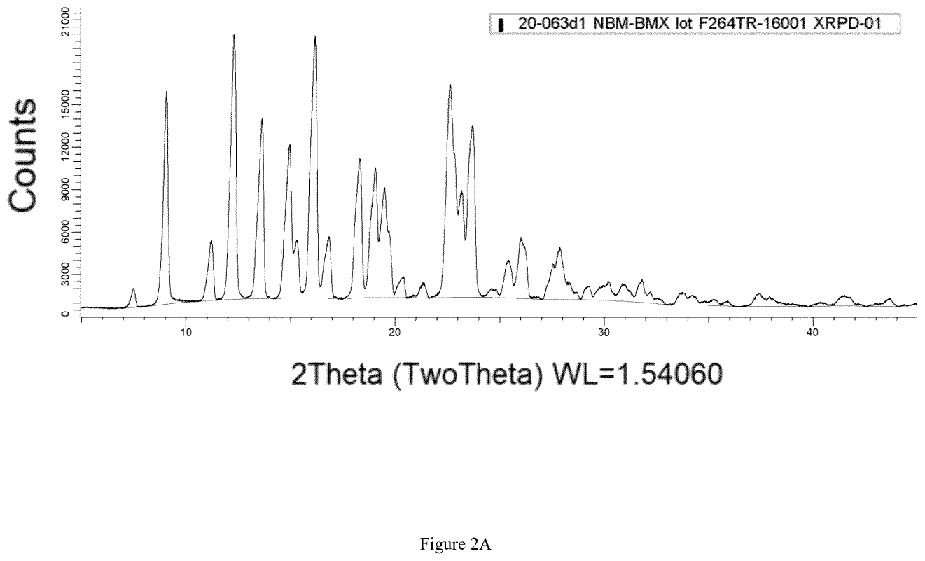

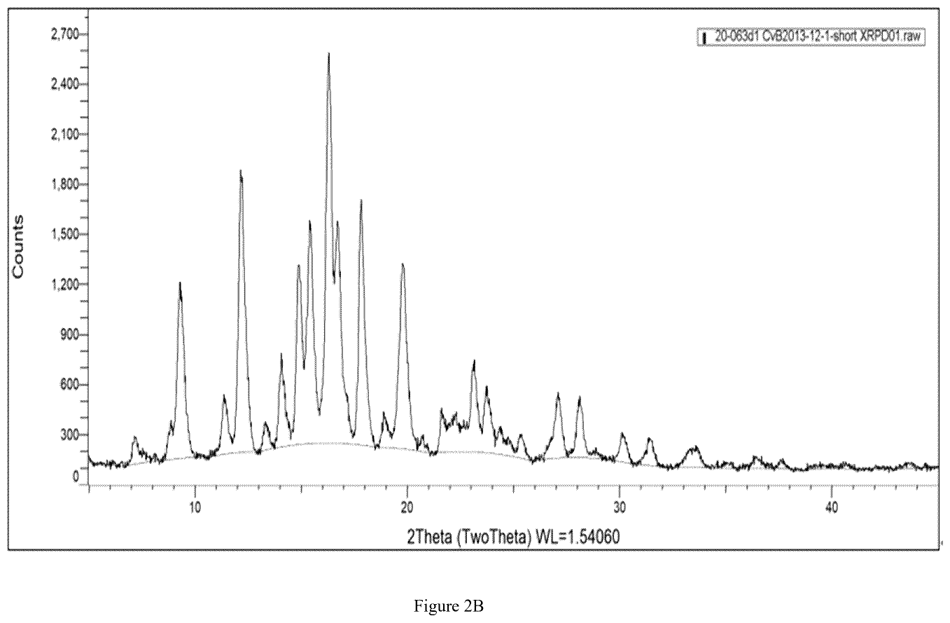

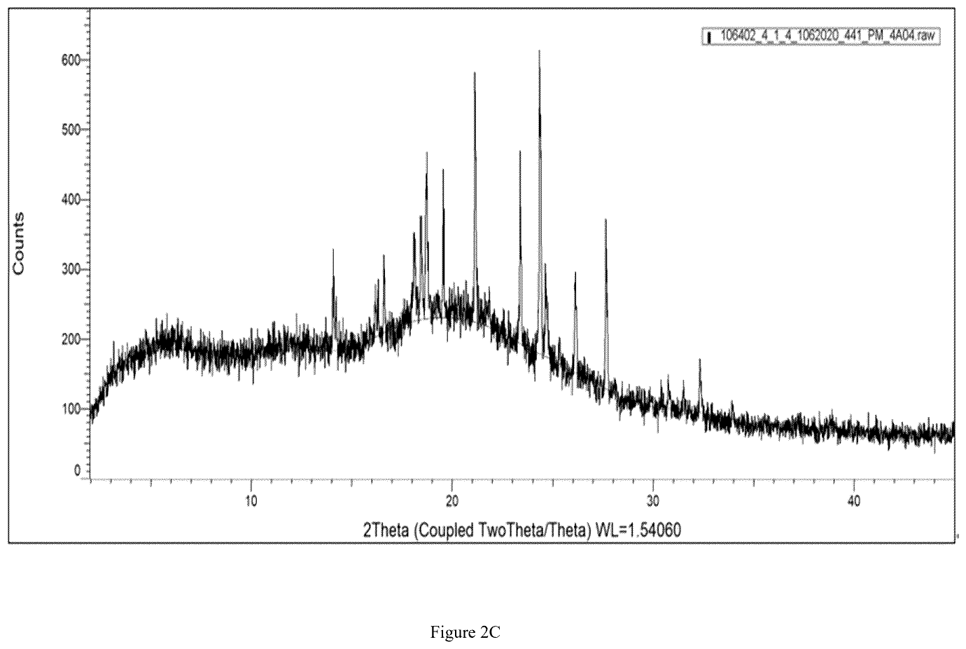

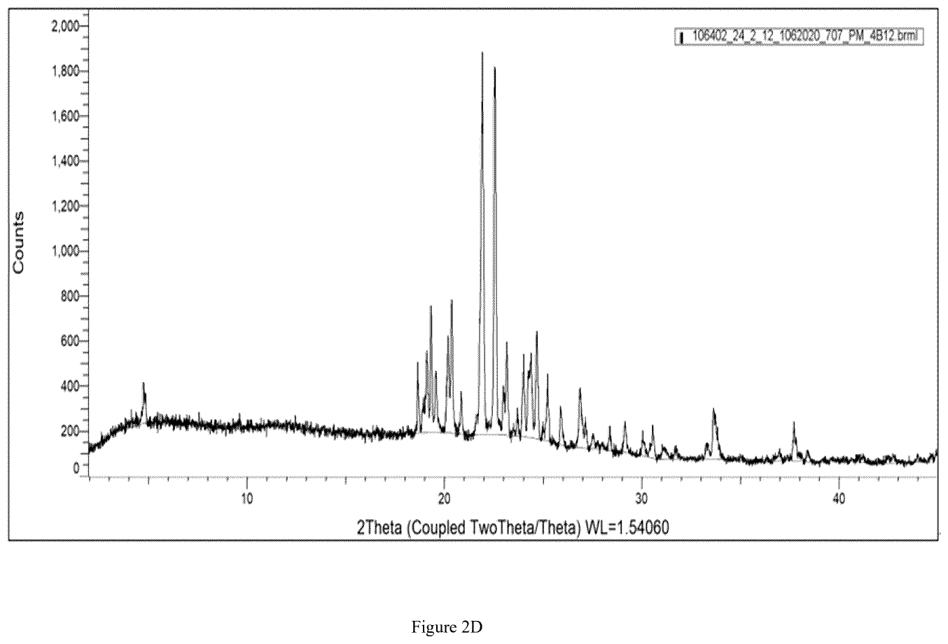

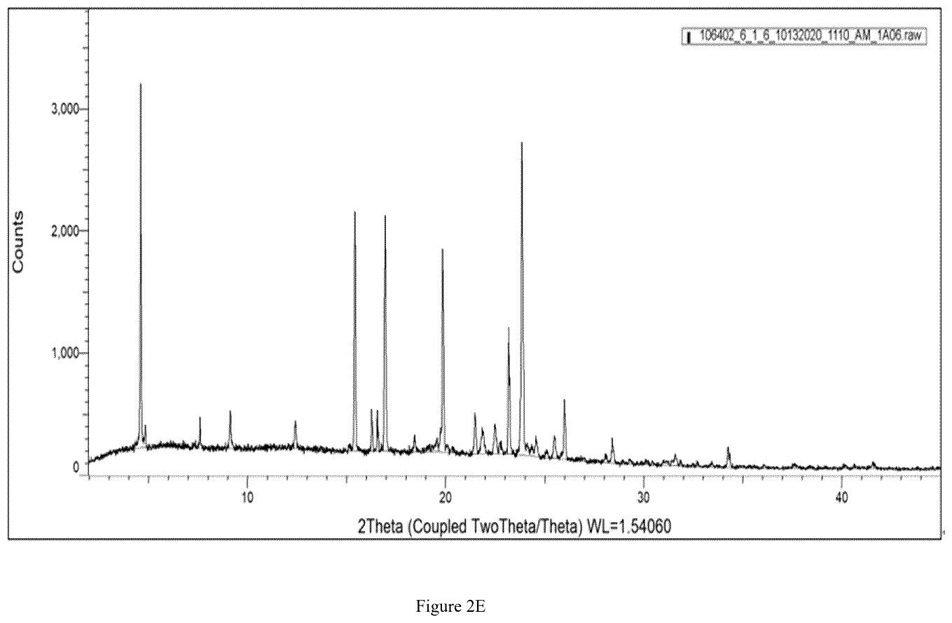

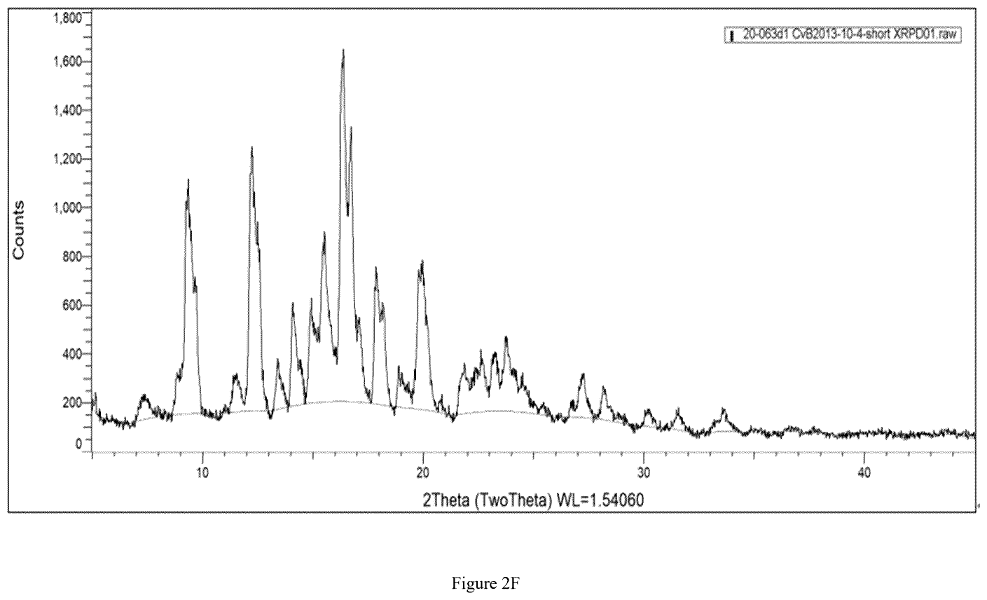

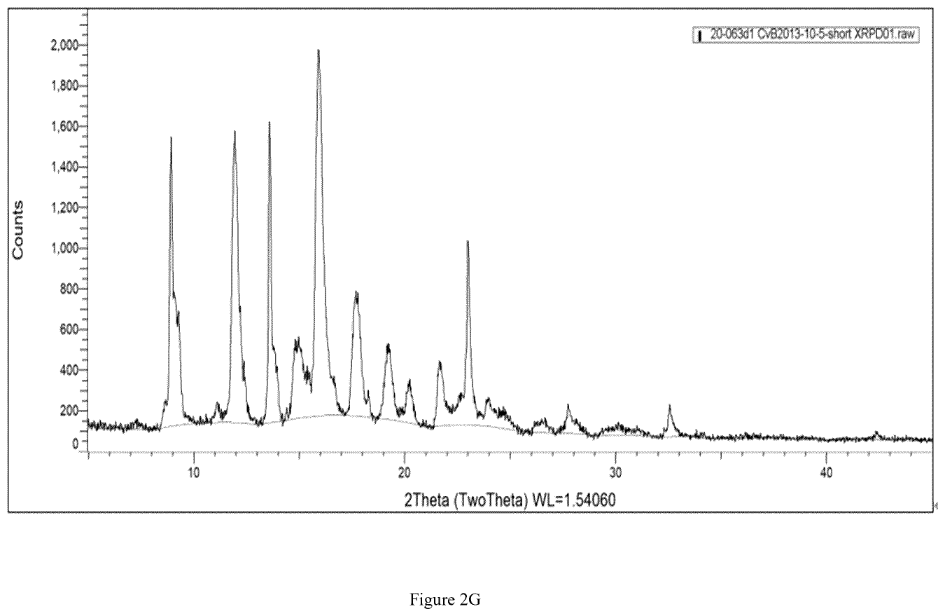

The crystal Form A of Compound BMX was characterized in X-ray powder diffraction method (XRPD) conducted by X-ray powder diffractometer Bruker AXS D2 PHASER in Bragg-Brentano configuration, equipment #1549 with a scan range of 5-45° 2θ. The X-ray source was a Cu anode at 30 kV, 10 mA (operating voltage and current). From beam to source, the slits that were used were a primary axial Soller slit 2.5°, a fixed divergence slit 1.0 mm (=0.61°), an 8.0 mm detector slit and a secondary axial Soller slit 2.5°. For monochromatisation, a Kβ-filter (0.5% Ni) was used. The detector was a linear detector LYNXEYE with receiving slit 5° detector opening. The sample stage was standard rotating (5/min) with beam stop. Measurement Centre v4.6. Data analysis was performed using Diffrac. Eva V4.1.1 evaluation software. No background correction or smoothing was applied to the patterns. The pattern result is shown in FIG. 2.

### Example 2. DSC Examination of Crystal Form A of Compound BMX

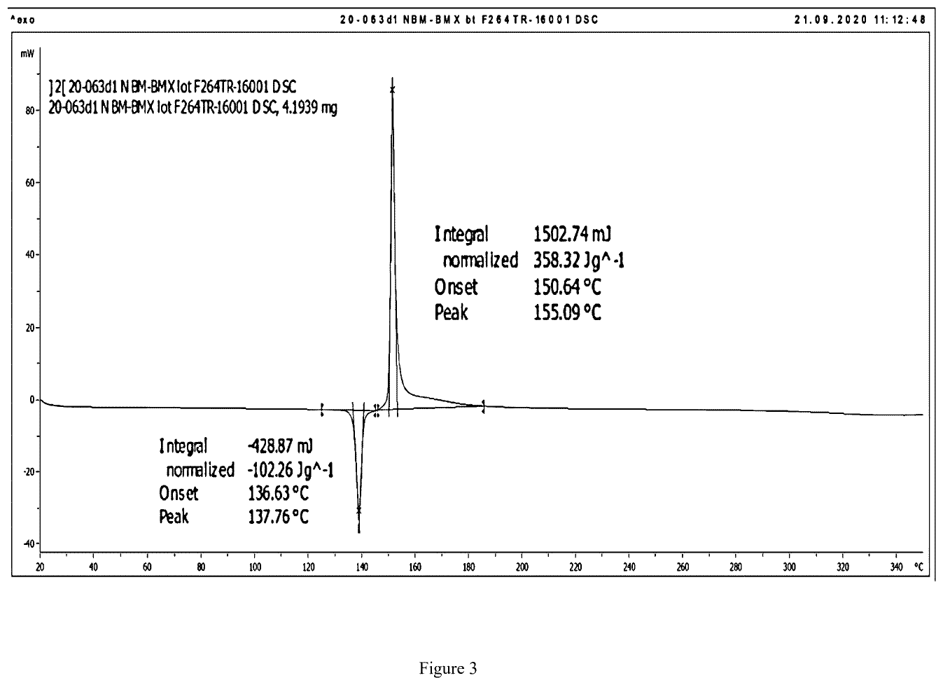

The DSC measurement was performed by using Mettler Toledo DSC-3+ equipment, with the sample heated from 20° C. to 350° C. in an aluminum (pierced) cup and a heating rate of 10° C./min being applied. Typically, 1-8 mg of sample was loaded onto a pre-weighed aluminum crucible and was kept at 20° C. for 5 minutes, after which it was heated at 10° C./min from 20° C. to 350° C. and kept at 350° C. for 1 minute. A nitrogen purge of 40 ml/min was maintained over the sample. The DSC thermogram of Crystal Form A of Compound BMX showed a sharp single melt at T peak=137.8° C., followed by a recrystallization event at T peak=155.1° C. as shown in FIG. 3. The software used for data collection and evaluation was STARe Software v15.00 build 8668, and no corrections were applied to the thermogram.

### Example 3. TGA Examination of Crystal Form A of Compound BMX

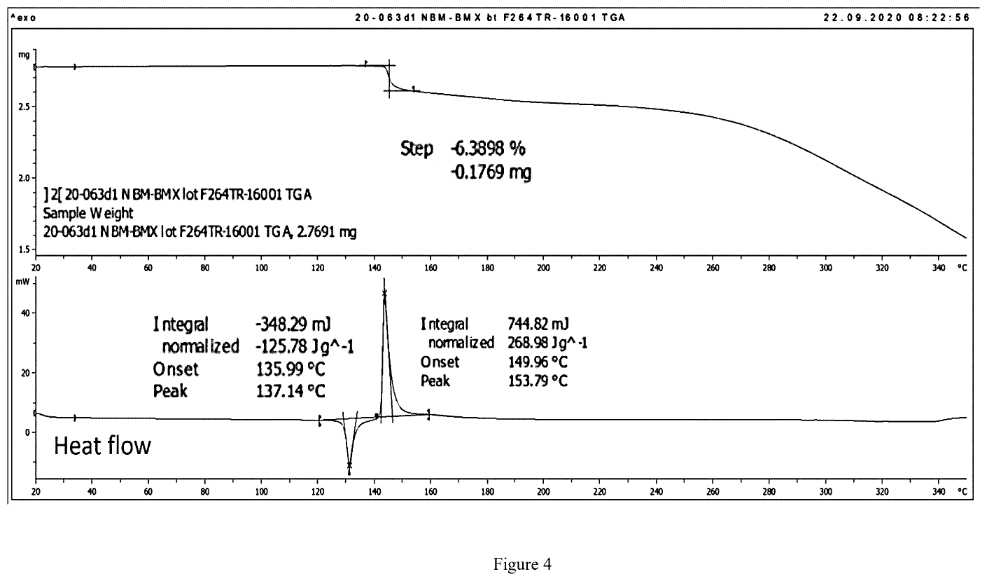

The TGA measurement was performed using Mettler Toledo TGA/DSC-3+, the same equipment DSC was applied with. Typically, 5-10 mg of sample was loaded into a pre-weighed aluminum crucible and was kept at 20° C. for 5 minutes, after which it was heated at 10° C./min from 20° C. to 350° C. A nitrogen purge of 40 ml/min was maintained over the sample. The data concluded that this form was an anhydrous/non-solvated form. A s shown in FIG. 4, it showed a mass loss of 6.4 wt-% during the recrystallization event which was an indication of decomposition.

### Example 4. Microscopy Study of Crystal Form A of Compound BMX

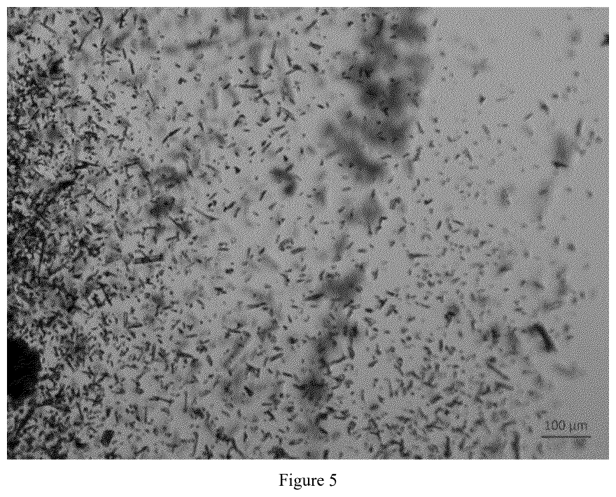

The microscopy image was obtained by a Zeiss microscope with four lenses: Zeiss A-Plan 5×/0.12, Zeiss A-Plan 10×/0.25, LD A-Plan 20×/0.30, and A chros TIGMAT 32×/0.40. The powder was transferred in the cavity of an object glass and a droplet of corn oil was added to evenly spread the sample. As shown in FIG. 5, the compound consisted of small needles with a length of about 20 μm. Data collection and evaluation were performed using Carl Zeiss Zen AxioVision Blue Edition Lite 2012 v1.0.0.0 software.

### Example 5. Hygroscopicity Determination

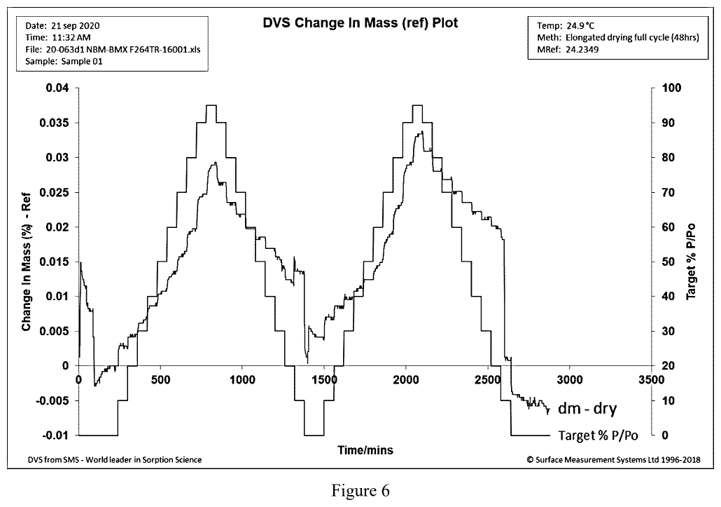

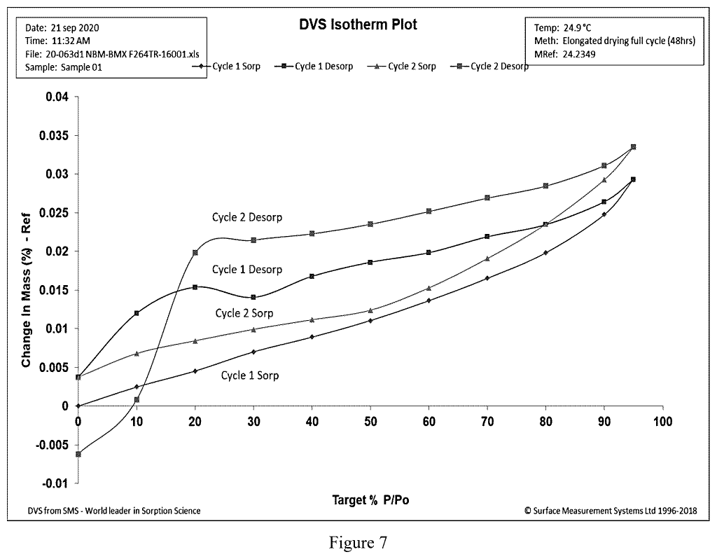

The hygroscopicity of the Crystal Form A of Compound BMX was determined using Surface Measurement Systems Ltd. DVS resolution equipment. The sample was loaded into balance pan, typically 10-30 mg, and equilibrated at 0% relative humidity (RH). When the material has dried, the RH is increased with 10% per step for 1 hour per increment, ending at 95% RH. After completion of the sorption cycle, the sample was dried using the same method. The DVS sorption and de-sorption plot was displayed in FIG. 6 and the DVS isotherm plot was displayed in FIG. 7. The compound showed a mass uptake of only 0.035 wt % and was therefore not hygroscopic. The software used for data collection is DVS Control Software for 21 CFR Part 11 v1.0.3.0. Data analysis is performed using DVS Standard Analysis Suite v7.2.0.9 (Standard).

Pre and post DVS analyses were conducted and the results of XRPD, DSC/TGA and microscopy did not show any differences after the DVS analyses compared to before. It means the compound is stable under these humid conditions.

### Example 6. Generation of Amorphous Material

The amorphous material of Compound BMX were obtained for the polymorph screen in order to avoid starting this screen with the thermodynamically stable polymorph, and to prevent the formation of less stable polymorphic forms.

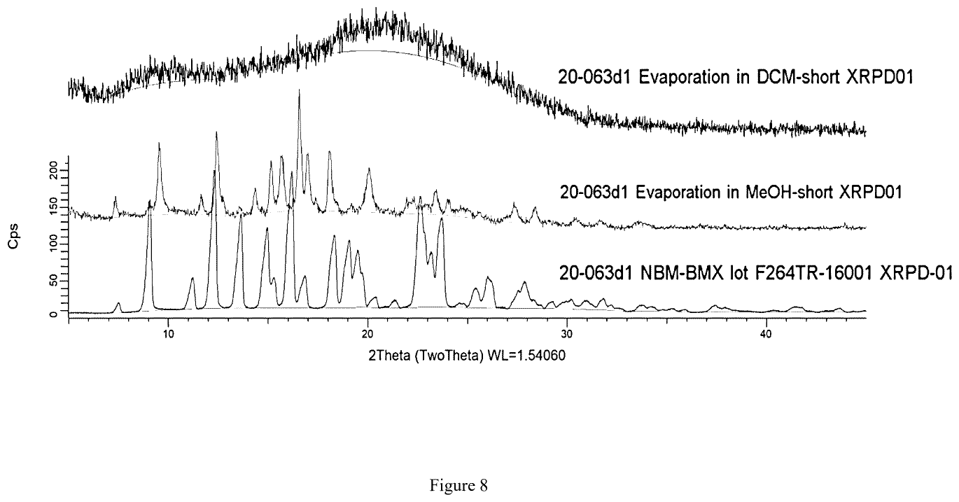

The amorphous material of Compound BMX was obtained by fast evaporation of solvents, solvents suitable for this technique are dichloromethane (DCM) and methanol (MeOH). Compound BMX was dissolved in dichloromethane and in methanol and both were evaporated to dryness under reduced pressure (0 mbar, RT). Afterwards, XRPD and TGA/DSC were measured. An XRPD overlay was displayed in FIG. 8. The experiment in DCM resulted in an amorphous pattern and was therefore also analyzed by TGA/DSC. The experiment that involved methanol resulted in a new XRPD, and was defined as Crystal Form B.

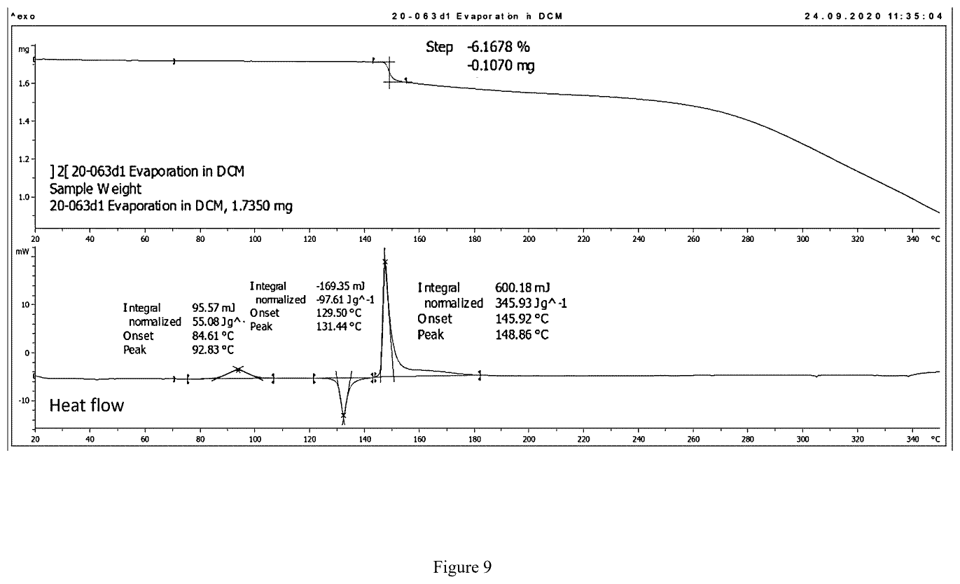

The TGA/DSC thermogram of the evaporation experiment in DCM was displayed in FIG. 9. The thermogram showed a recrystallization event at T peak=93° C. followed by the original melt (T peak=131° C.) and recrystallization event. The recrystallization event indicated that the amorphous material recrystallized into the Crystal Form A.

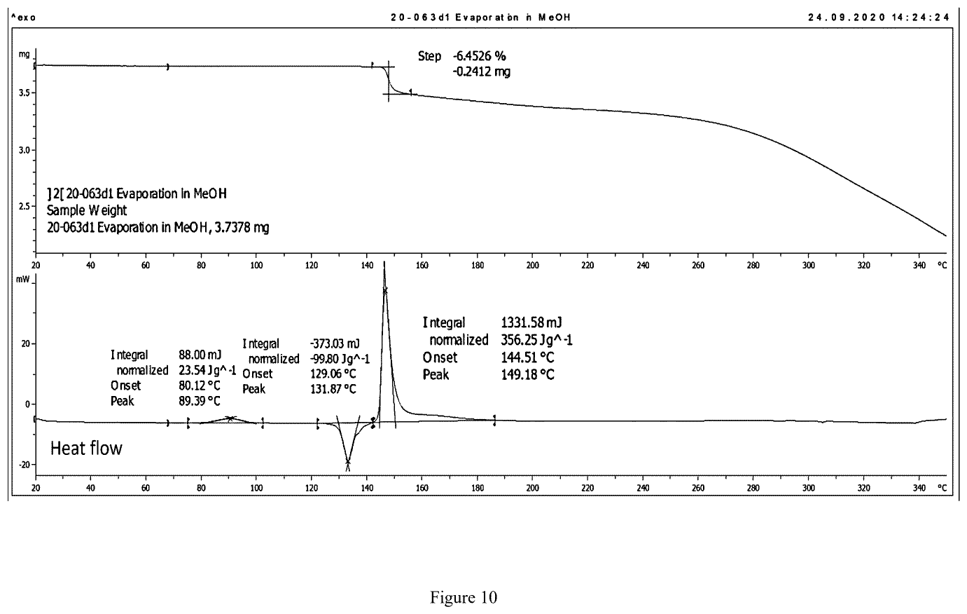

The TGA/DSC thermogram of the evaporation experiment in MeOH is displayed in FIG. 10. Since this experiment resulted in a new form, it was expected that it showed changes in the TGA/DSC thermogram. Same as the experiment in DCM, it showed a recrystallization event at T peak=89° C. followed by the original melt (T peak=132° C.) and recrystallization event. This indicated that the new form recrystallized into Crystal Form A before the melt.

### Example 7. Solubility Study

The solubility of amorphous material of Compound BMX was determined by the shake flask method. The amorphous material was obtained by dissolving 500 mg of the Crystal From A of Compound BMX in 10 mL dichloromethane, this solution was evaporated to dryness in vacuum conditions (0 mbar, RT).

The solvents for the solubility determination were selected based on the Principal Component Analysis (PCA) of properties, which means that some pharmaceutically unattractive solvents were included to create a better understanding of the parameter space. The Principal Component Analysis focusses on polarity, polarizability and hydrogen bonding.

The listed solvents were added stepwise to 10 mg of the compound, with 15 minutes in between additions, until complete dissolution was obtained, or a solubility of less than 10 mg/ml was reached. Experiments that showed a solubility of less than 10 mg/mL were stored in the shaker for 24 hours. Subsequently, the samples were filtered and diluted for LC analysis and the concentration is determined using a calibration line. An overview of the results is given in Table 1.

TABLE 1

| | Solubility | | |
|---|---|---|---|
| Solvent | (mg/mL) | USP classification | XRPD |
| 2-Ethly-1-butanol | 8.8 | Slightly soluble | Amorphous |
| Acetonitrile | 10-33 | Sparingly soluble | Amorphous |
| Methanol | 31-94 | Soluble | Form B |
| Water | 0.0 | Practically insoluble | Form B |
| Dimethyl sulfoxide | 31-94 | Soluble | Gel |
| Ethyl sulfoxide | 6.9 | Slightly soluble | Form A |
| Methyl-tert-butyl ether | 3.3 | Slightly soluble | Form A |
| n-Heptane | 0.0 | Practically insoluble | Form B |
| Ethyl butyl ketone | 10-33 | Sparingly soluble | Form B |
| Anisole | 9.7 | Slightly soluble | Form B |

The remaining solids, and solids obtained after complete evaporation of a clear solution, were analyzed by XRPD in order to get a first indication of the polymorphic behavior of the compound. Solvent selection for the polymorph study was based on these results. In three cases (ethyl salicylate, MtBE and anisole) immediate crystallization was observed. Only one new XRPD pattern was observed and is defined as Crystal Form B of Compound BMX. The Crystal Form B was found in water, methanol, ethyl butyl ketone, anisole and heptane. This is the same pattern/form as found after the evaporation experiment in order to obtain the amorphous material.

### Example 8. Polymorph Study

The polymorph study was conducted using the following crystallization techniques:
1. slurry at 50° C. for 2 hours;
2. cooling crystallization, 50° C.-10° C.;
3. evaporation;
4. precipitation; and
5. shake slurry at room temperature for 16 hours (amorphous material).

The shake slurry experiments at room temperature were performed using amorphous Compound BMX, the slurry at 50° C., cooling, anti-solvent and evaporative crystallization were performed using the Crystal Form A. Sixteen different solvents/solvent combinations were used. These were selected based upon their wide variety in PCA properties and the results of the solubility study. Some pharmaceutically unattractive solvents were included to create a better understanding of the parameter space. The selected solvents are listed in Table 2. The solvents/co-solvents were added in the following ratio's: 100/0, 80/20, 60/40, 40/60, 20/80 and 5/95.

TABLE 2

| | ICH | PCA | | ICH | PCA | Anti- | ICH | PCA |
|---|---|---|---|---|---|---|---|---|
| Solvent | Class | (#) | Co-solvent | Class | (#) | solvent | Class | (#) |
| DMSO | 3 | 3 | Ethanol | 3 | 1 | Water | — | 2 |
| Acetonitrile | 2 | 1 | Ethanol | 3 | 1 | Water | — | 2 |
| 1,3-Dioxolane | — | 6 | Ethanol | 3 | 1 | Water | — | 2 |
| Methanol | 3 | 1 | Ethanol | 3 | 1 | Water | — | 2 |
| 2-Butanone | 3 | 1 | Cyclohexane | 2 | 6 | n-Heptane | 3 | 6 |
| CPME | — | 6 | Cyclohexane | 2 | 6 | n-Heptane | 3 | 6 |
| MIBK | 2 | 5 | Cyclohexane | 2 | 6 | n-Heptane | 3 | 6 |
| Ethyl butyl ketone | — | 7 | Cyclohexane | 2 | 6 | n-Heptane | 3 | 6 |
| Ethyl salicylate | — | 4 | Tetrahydrofuran | 2 | 5 | n-Heptane | 3 | 6 |
| Acetic acid | 3 | 2 | Tetrahydrofuran | 2 | 5 | n-Heptane | 3 | 6 |
| 2-Butanol | 3 | 1 | Tetrahydrofuran | 2 | 5 | n-Heptane | 3 | 6 |
| Pyridine | 2 | 3 | Tetrahydrofuran | 2 | 5 | n-Heptane | 3 | 6 |
| Ethyl acetate | 3 | 5 | Toluene | 2 | 8 | n-Heptane | 3 | 6 |
| 2-MethylTHF | — | 5 | Toluene | 2 | 8 | n-Heptane | 3 | 6 |
| Hexafluoro benzene | — | 8 | Toluene | 2 | 8 | n-Heptane | 3 | 6 |
| Anisole | 3 | 8 | Toluene | 2 | 8 | n-Heptane | 3 | 6 |

The amorphous material of Compound BMX was prepared by making a stock solution in dichloromethane: 1 gram in 20 mL. 0.2 mL of this solution was dispensed in each well of the shake slurry plate and the dichloromethane was allowed to evaporate at ambient conditions. The selected solvents and co-solvents (400 μl in total) were added in the following ratio's; 100-0, 80-20, 60-40, 40-60, 20-80 and 5-95. The plate was stored on a shaker for 24 hours. Remaining solvents were absorbed by filter paper, thereafter the plate was stored in vacuum (0 mbar, RT) in order to dry the obtained solids.

Thirty (30) mg of Crystal Form A of Compound BMX was dispensed in each well of the master plate (slurry at 50° C.) by the Unchained Labs Protege equipment. Selected solvents and co-solvents (800 μl in total) were added in the following ratio's; 100-0, 80-20, 60-40, 40-60, 20-80 and 5-95. The plate was stored on a thermoshaker, 50° C., 400 rpm. After 2 hours, the clear liquids were divided over the cooling plate, anti-solvent plate and evaporation plate, 200 μl each. The remaining solvents were absorbed by filter paper, there after the plate was stored in vacuum (0 mbar, RT) in order to dry the obtained solids.

The anti-solvent plate was pre-dispensed with 400 μl of anti-solvent. After the addition of the clear liquids from the master plate, the anti-solvent plate was stored at room temperature for 16 hours in order to obtain precipitation. The remaining liquids were absorbed by filter paper there after the plate was stored in vacuum (0 mbar, RT) in order to dry the obtained solids. The cooling plate was cooled from 50° C. to 5° C. and stored at 5° C. for 16 hours in order to induce crystallization. The remaining liquids were absorbed by filter paper there after the plate was stored in vacuum (0 mbar, RT) in order to dry the obtained solids. The evaporation plate was stored in a vacuum oven at reduced pressure, 0 mbar, RT. The wells of all plates analyzed by XRPD as shown in Table 3.

TABLE 3

| Solvent/ | Slurry | | | | Shake |
|---|---|---|---|---|---|
| co-solvent | SO*C | Cooling | Evaporation | Precipitation | slurry |
| DMSO/ethanol | Form A | — | — | Form A | — |
| | Form C | | | | |
| Acetonitrile/ | Form A | — | Form A | Form A | Form A |
| ethanol | Form B | | Form B | | Form B |
| | | | Form D | | Form D |
| 1,3-Dioxolane/ | Form A | — | Form D | Form D | Form B |
| ethanol | Form B | | | | |
| Methanol/ | Form A | — | Form D | — | Form A |
| ethanol | Form B | | | | |
| 2-Butanone/ | Form A | — | — | Form A | Form A |
| cyclohexane | | | | Form D | Form D |
| CPME/ | Form A | — | Form A | Form A | Form D |
| cyclohexane | | | | Form D | |
| MIBK/ | Form A | — | Form D | Form D | Form D |
| cyclohexane | | | | | |
| Ethyl butyl | Form A | Form A | Form A | Form D | Form D |
| ketone/ | | | | | |
| cyclohexane | | | | | |
| Ethyl | Form A | — | Form D | Form A | Form B |
| salicylate/THF | | | | | |
| Acetic acid/ | Form A | — | Form B | Form A | — |
| THF | Form B | | Form D | | |
| | Form D | | | | |
| 2-Butanol/THF | Form A | — | Form D | Form A | — |
| | Form D | | | | |
| Pyridine/THF | — | — | Form D | Form A | — |
| Ethyl acetate/ | Form A | Form A | Form A | Form A | Form A |
| toluene | | | | Form D | Form B |
| | | | | | Form D |
| 2-MethylTHF/ | Form A | Form A | Form B | Form D | Form A |
| toluene | Form B | | | | Form D |
| | Form D | | | | |
| Hexafluoro | Form A | Form A | Form A | Form A | Form D |
| benzene/ | | | | | |
| toluene | | | | | |
| Anisole/toluene | Form A | — | — | — | — |

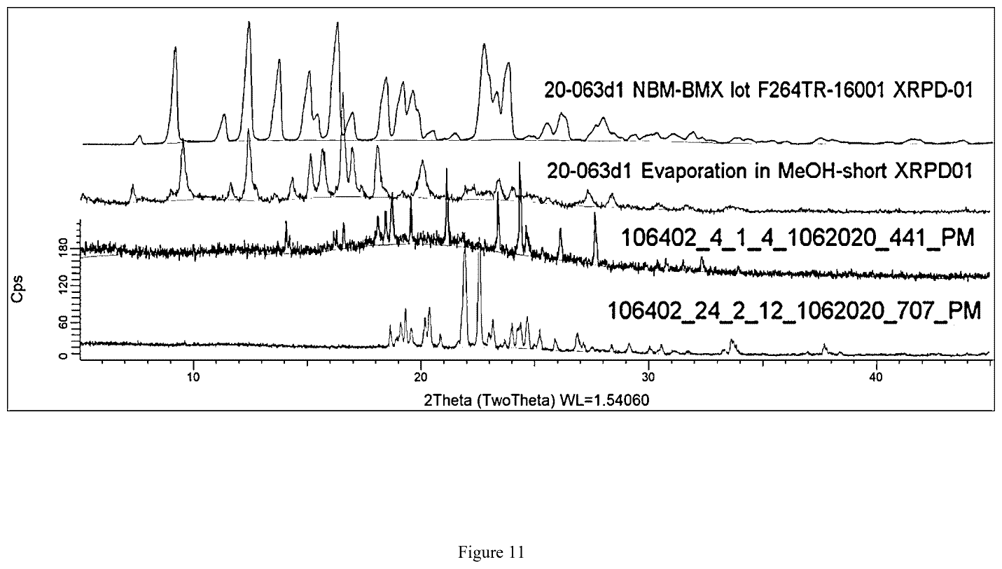

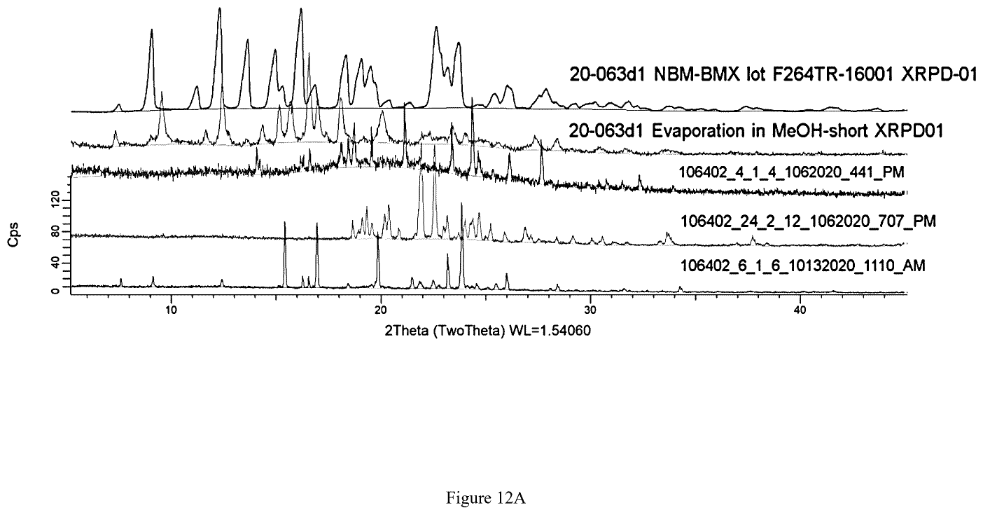

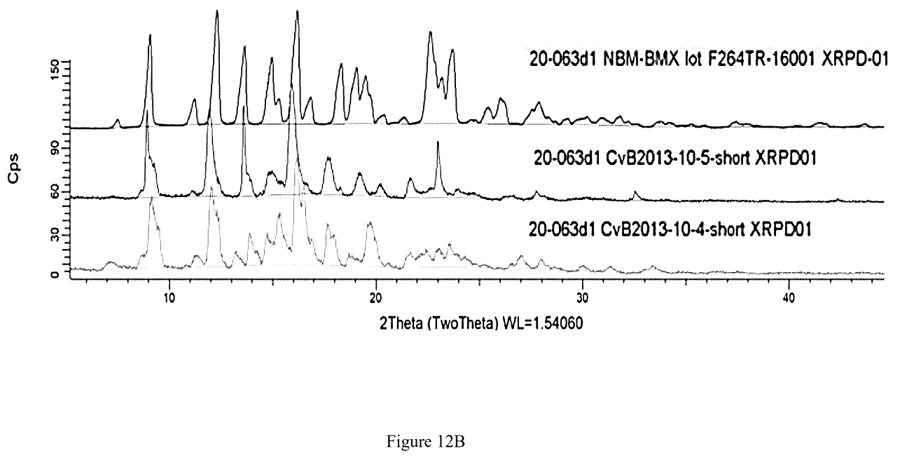

Four different forms were observed, including Crystal Forms A, B, C and D. The experiments in the shake slurry plate at room temperature, using amorphous material, resulted in mainly Crystal Form D. On the contrary, the experiments in the slurry plate at 50° C., using Crystal Form A, remained mostly as the Crystal Form A. This indicated that there was a temperature effect and that Crystal Form A was very likely the most stable polymorphic form. Both Crystal Form A and Crystal Form D were found in different solvent combinations using different crystallization techniques. Crystal Form B was observed in different solvent combinations, mostly in the ones using ethanol, tetrahydrofuran and toluene. Crystal Form C was only observed once, being found in a slurry experiment at 50° C. in DMSO/Ethanol (60/40-%). An XRPD overlay of Crystal Form A, Crystal Form B, Crystal Form C and Crystal Form D was displayed in FIG. 11. All plates were stored at 40° C./75% RH for 4 days in order to determine the stability of the polymorphic forms. Cystal Form C was not a stable form and converted into polymorphic Crystal Form D under these conditions. An amorphous solid converted into a new polymorphic form: Crystal Form E (slurry experiment at 50° C. in ethanol). An overlay of the five forms was displayed in FIG. 12A.

### Example 9. Scale Up Experiment for Reproducibility Check

The experiments that resulted in a new form were reproduced at 100 mg scale with Crystal Form A in order to check reproducibility, scalability and to have sufficient material for full characterization. The slurry experiments were kept at 50° C. for 16 hours, 500 rpm, and cooled to room temperature. While conducting the Anti-solvent experiments, compound was dissolved at 50° C. and added into another vial containing the anti-solvent, and when conducting evaporation experiments, compound was dissolved at 50° C. and solvents were evaporated in vacuum, 0 bar, RT. The experimental details and XRPD, DSC results are displayed in Table 4.

TABLE 4

| Form to | | | | | Melt by |
|---|---|---|---|---|---|
| repro- | | Crystallization | | | DSC |
| duce | Solvent | technique | Appearance | XRPD | (*C) |
| — | — | — | — | Form A | 137.1 |
| B | 2-MeTHF | Slurry 50° C., | Clear | Poor | 131.5 |
| | (1 mL) | 16 hours | solution, | crys- | 136.6 |
| | | | evaporated | talline | |
| B | MeOH/EtOH | Slurry 50° C., | White | Form A | 137.5 |
| | (60/40) | 16 hours | powder | | |
| | (1 mL) | | | | |
| C | DMSO/ | Slurry 50° C., | Clear | Oil - no | No data |
| | EtOH | 16 hours | solution, | data | |
| | (60/40) | | evaporated | | |
| | (1 mL) | | | | |
| D | 1,3- | Anti-solvent | White | Form F | 133.5 |
| | Dioxolane | Water (2 mL) | needles | | 139.2 |
| D | MIBK | Anti-solvent | White | Form G | 132.6 |
| | (4 mL) | Heptane | needles | | |
| | | (2 mL) | | | |
| E | Ethanol(1 | Slurry 50° C., | White | Form A | 137.4 |
| | mL) | 16 hours | powder | | |
| B | Methanol(3 | Evaporation | White | Form B | 131.4 |
| | mL) | | powder | | |
| D | MIBK | Evaporation | White | Form A | No data |
| | (5 mL) | | powder | | |

As the above table describes, only Crystal Form B was reproducible, some other conditions resulted in new forms. In order to characterize all the forms by TGA/DSC, the samples from the screen were taken, results are displayed in Table 5.

TABLE 5

| | Crystallization | | Melt by DSC |
|---|---|---|---|
| Solvent | technique | XRPD | (° C.) |
| 2-MeTHF/toluene (5/95) | Shake slurry, RT | Form D | 133.0 |
| DMSO/ethanol (5/95) | Slurry, 50° C. | Form E | 127.1 |

No TGA/DSC thermogram of Crystal Form C is available since that form was found unstable. The melting events of the other forms were all lower than the melting event of Crystal Form A, ranging from 131° C.-139° C. Form E was a mixture, with a melt at 133.5° C. and at 139.2° C. The fact that Crystal Form A had the highest melting event indicates that this form was the most stable polymorphic form. As described above, scale up of Crystal Form C, D and E was not successful. Therefore, the experiments were repeated using the exact same conditions as used in the screening (30 mg scale). None of the forms was reproducible at this scale. Crystal Form D was observed on multiple occasions but seems to be irreproducible. The Crystal Form C and Form E were only observed once in the screening and were not stable forms. The fact that all experiments remained as Cystal Form A indicated that this form was very stable and most suitable for further development.

### Example 10. Determination of the Most Stable Polymorphic Form

To confirm the theory that Crystal Form A was the most stable form, competitive slurry experiments were performed. No material of Crystal Form C, D, or E was available since these could not be reproduced. The competitive slurry was performed using Crystal Forms B, E, F and poorly crystalline material, with 10 mg of each form being added in two 1 mL vials.

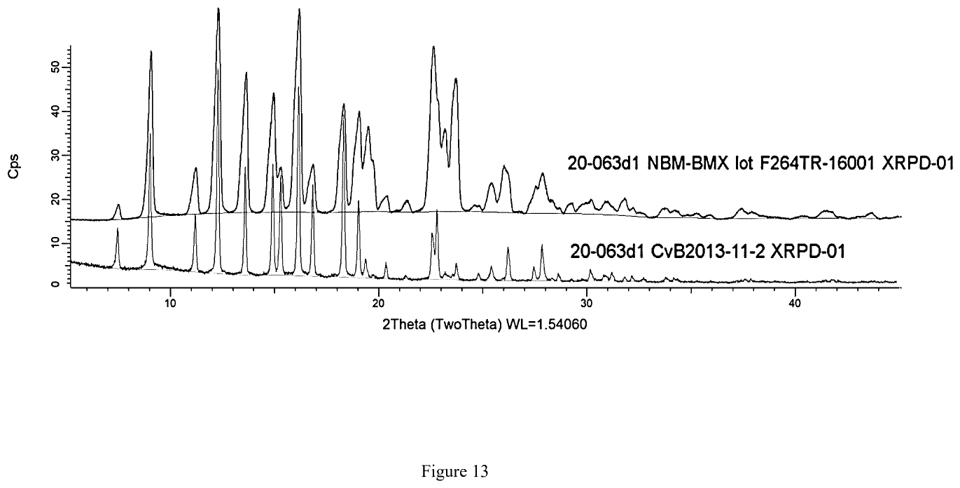

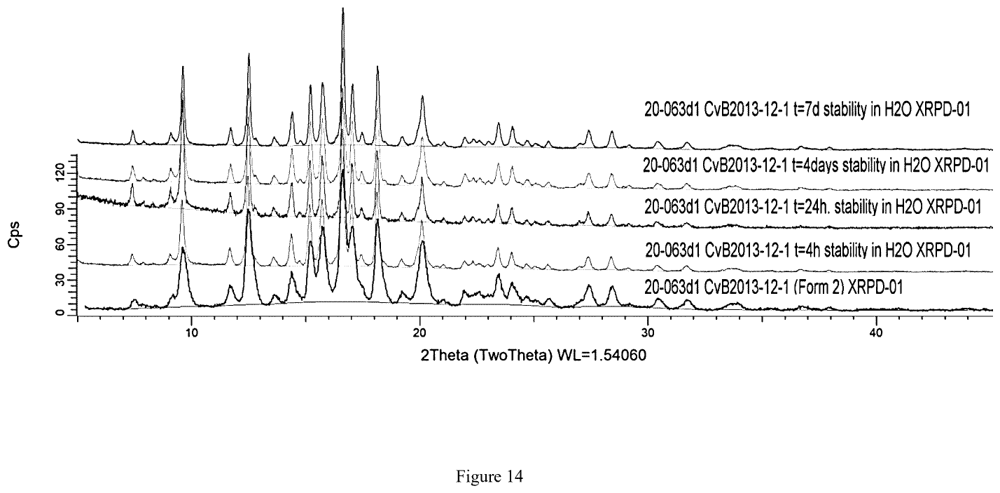

The competitive slurry experiment in 2-methyl tetrahydrofuran gave a clear solution and the experiment in 2-propanol gave a white slurry. The white powder was characterized in XRPD and Crystal Form A was found as shown in FIG. 13. This indicates that Cystal Form A is the thermodynamically most stable form.

### Example 11. Aqueous Solubility of Crystal Form A of Compound BMX

The Crystal Form A was determined as most stable polymorphic form. The solubility of this form was tested in the process solvents (Ethyl acetate and hexane) and in aqueous buffer systems. 100 mg of Crystal Form A of Compound BMX was weighed into 4 mL vials with 2 mL of the specified solvent/aqueous buffer system being added. These solutions were shaken at room temperature for 24 hours. The pH was monitored in time and remained in the correct range. Samples were filtered and diluted (5× in ACN/MeOH 1/1) and measured using H PLC. The concentration of the samples is determined using a calibration line. The results are displayed in Table 6.

TABLE 6

| | Concentration | | Area | Dilution | Injection | Solubility | |
|---|---|---|---|---|---|---|---|
| Solvent | (mg/mL) | PH | (mAU*sec) | factor | volume | (mg/mL) | XRPD |
| Ethyl acetate | 50 | — | 28834.0 | 5 | 2 | 20.78 | Form A |
| Hexane | 50 | — | 0 | 5 | 2 | 0.00 | Form A |
| Water | 50 | 7 | 0 | 5 | 2 | 0.00 | Form A |
| FaSSGF | 50 | 1.2 | 0 | 5 | 2 | 0.00 | Form A |
| FaSSIF | 50 | 6.5 | 20.4 | 5 | 2 | 0.01 | Form A |
| FeSSIF | 50 | 5.8 | 246.0 | 5 | 2 | 0.18 | Form A |
| Phosphate | 50 | 7.4 | 0 | 5 | 2 | 0.00 | Form A |
| buffer pH | | | | | | | |
| 7.4 | | | | | | | |

Form A was insoluble in Hexane, water, FaSSGF, FASSIF and phosphate buffer pH 7.4. It did have some solubility in FeSSIF (0.18 mg/mL) and was soluble in process solvent ethyl acetate (20.8 mg/mL). No changes in polymorphic form were observed.

### Example 12. Stability Test of Form B

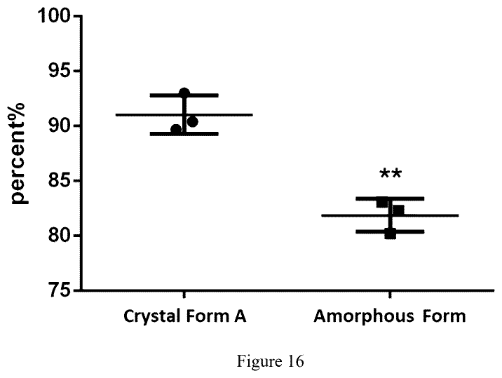

No changes were observed in the XRPD with the Crystal Form B of Compound BMX being slurred in water at room temperature and taken at different timepoints: 4 hours, 24 hours, 4 days and 7 days, as shown in FIG. 16. This indicated that once Crystal Form B had been formed it was very stable in water.

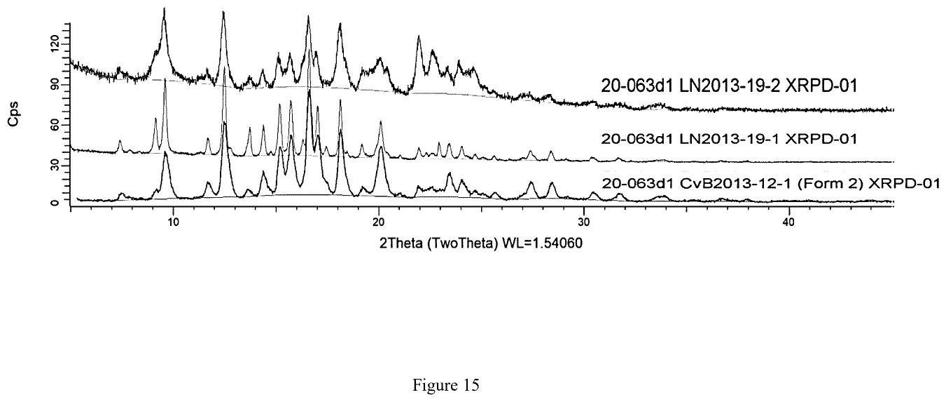

A small portion of seeds of Crystal Form A was added and slurring at 50° C.; however, Form B could not be converted into Crystal Form A, as shown in FIG. 15. Accordingly, it was proved once again that Crystal Form B of Compound BMX was stable in water.

### Example 13. Comparison in Stability Between the Amorphous and the Crystal Forms

The amorphous Form and the Crystal Form A of Compound BMX samples were exposed at a cool white fluorescent lamp that has an output similar to that specified by ISO 10977 and UV fluorescent lamp with a spectral distribution from 320 nm to 400 nm for evaluating the photo stress stability. As shown in FIG. 16, Crystal Form A of Compound BMX showed 10% degradation, while the amorphous form showed 20% degradation after 48 hours of exposure to photo-stress conditions. The results demonstrated that the Crystal Form A was significantly more stable than the amorphous form under stress conditions.

While this specification contains many specifics, these should not be construed as limitations on the scope of the invention or of what may be claimed, but rather as descriptions of features specific to particular embodiments or examples of the invention. Certain features that are described in this specification in the context of separate embodiments or examples can also be implemented in combination in a single embodiment.

What is claimed is:

1. A Crystal Form of Compound BMX having the chemical structure below, 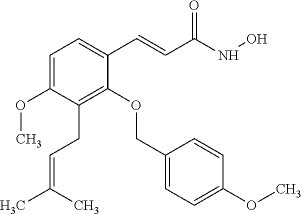 [SMILES: COc1ccc(COc2c(/C=C/C(=O)NO)ccc(OC)c2CC=C(C)C)cc1]
which is selected from the group consisting of Crystal Form A, Crystal Form B, Crystal Form C, Crystal Form D, Crystal Form E, Crystal Form F and Crystal Form G.

2. The Crystal Form of Compound BMX of claim 1, which is the Crystal Form A characterized in X-ray powder diffraction (XRPD) pattern having characteristic peaks at 2θ of about 4.58°, 7.58°, 9.14°, 11.30°, 12.41°, 13.71°, 15.05°, 15.41°, 16.27°, 16.97°, 18.44°, 19.16°, 19.51°, 19.87°, 20.49°, 22.71°, 22.92°, 23.33°, 23.86°, 24.92°, 25.55°, 26.36°, 27.58°, 28.00°, 28.48°, 28.77°, 29.38°, and 30.32°; and an infrared spectrum having characteristic absorption peaks at about 3250, 2909, 2837, 1643, 1590, 1515, 1484, 1462, 1250, 1173, 1088, 991, 823, 806, 796, 516 and 481 cm⁻¹.

3. The Crystal Form of Compound BMX of claim 2, in which the Crystal Form A is characterized in a differential scanning calorimetry (DSC) pattern having a sharp single melt at 137.8° C. and a recrystallization event at 155.1° C.

4. A method for preparing the Crystal Form A set forth in claim 2, comprising the steps of:
dissolving Compound BMX in ethyl acetate to obtain a solution;
slowly adding n-hexane with stirring and cooling it to a room temperature to obtain a solution; and
cooling the solution to 0˜5° C. for accelerating precipitation to obtain the Crystal Form A.

5. The Crystal Form of Compound BMX of claim 1, which is the Crystal Form B characterized in an X-ray powder diffraction (XRPD) pattern having peaks at 2θ of about 7.19°, 8.86°, 9.36°, 11.40°, 12.17°, 13.32°, 14.08°, 14.88°, 15.43°, 16.30°, 16.72°, 17.83, 18.91°, 19.81°, 23.13°, 23.75°, 27.01°, 28.13°, 30.10°, and 31.42°.

6. The Crystal Form of Compound BMX of claim 5, in which the Crystal Form B is characterized in a differential scanning calorimetry (DSC) pattern having a sharp single melt at 132° C. and a recrystallization event at 89° C.

7. A method for preparing the Crystal Form B set forth in claim 5, comprising the steps of:
dissolving Compound BMX in dichloromethane and/or in methanol to obtain a solution;
fast evaporating the solution obtained in the above step to obtain an amorphous material of Compound BMX; and
redissolving the amorphous material of Compound BMX in MeOH and water to obtain the Crystal Form B.

8. The Crystal Form of Compound BMX of claim 1, which is Crystal Form C characterized in an X-ray powder diffraction (XRPD) pattern having peaks at 2θ of about 14.08°, 16.17°, 16.30°, 16.60°, 18.08°, 18.43°, 18.71°, 19.55°, 21.13°, 23.38°, 24.35°, 24.64°, 26.13°, 27.65°, and 32.33°.

9. The Crystal Form of Compound BMX of claim 8, in which the Crystal Form C is characterized in an X-ray powder diffraction (XRPD) pattern having peaks at 2θ of about 4.77°, 18.65°, 19.10°, 19.32°, 19.55°, 20.17°, 20.18°, 20.36°, 20.84°, 21.92°, 22.54°, 22.98°, 23.16°, 24.02°, 24.25°, 24.38°, 24.67°, 25.23°, 25.89°, 26.86°, 33.64°.

10. The Crystal Form of Compound BMX of claim 1, which is the Crystal Form E characterized in an X-ray powder diffraction (XRPD) pattern having peaks at 2θ of about 4.61°, 7.60°, 9.13°, 12.40°, 15.42°, 16.27°, 16.56°, 16.95°, 19.85°, 21.49°, 21.84°, 22.48°, 23.19°, 23.85°, 24.56°, 25.49°, 25.99°, 28.42°, and 34.26°.

11. The Crystal Form of Compound BMX of claim 1, which is Crystal Form F characterized in an X-ray powder diffraction (XRPD) pattern having peaks at 2θ of about 7.32°, 9.37°, 11.47°, 12.23°, 13.41°, 14.07°, 14.93°, 15.54°, 16.38°, 16.74°, 17.07°, 17.86°, 18.18°, 18.90°, 19.90°, 21.87°, 22.61°, 23.21°, 23.72°, 27.18°, and 28.16°.

12. The Crystal Form of Compound BMX of claim 1, which is Crystal Form G characterized in an X-ray powder diffraction (XRPD) pattern having peaks at 2θ of about 8.92°, 9.27°, 11.94°, 13.58°, 13.78°, 14.94°, 15.92°, 17.68°, 19.21°, 20.19°, 21.65°, 22.99°, 27.75°, and 32.55°.

13. A pharmaceutical composition, comprising the Crystal Form of Compound BMX of claim 1, and a pharmaceutically acceptable carrier.
